(19) United States
(12) Plant Patent Application Publication
(10) Pub. No.: US 2026/0283060 P1
(43) Pub. Date: Sep. 17, 2026

(54) MEXICAN LIME TREE NAMED 'LILAC LIME'

(50) ;

(71) Applicant: The United States of America, as represented by the Secretary of Agriculture, Washington, DC (US)

(72) Inventors: JAMES G. THOMSON, EL CERRITO, CA (US); MIN SHAO, NEWARK, CA (US)

(21) Appl. No.: 19/077,456

(22) Filed: Mar. 12, 2025

(51) Int. Cl. A01H 6/78; A01H 5/08

(52) U.S. Cl. CPC A01H 6/785; A01H 5/08
USPC PLT/201

(57) ABSTRACT

A new and distinct lime cultivar ‘Lilac Lime’, that originated from the Mexican lime tree. This new lime cultivar can be distinguished by its fruit flesh which contains anthocyanin and is a deep purple color while retaining the green peel.

Latin name of the genus and species of the plant claimed: ‘Lilac Lime’ is a Mexican lime tree that is a Citrus aurantiifolia.

Variety denomination: The new Mexican lime tree is of the variety denominated ‘Lilac Lime’.

### BACKGROUND OF THE NEW PLANT

The present invention relates to a new and distinct Mexican lime cultivar designated ‘Lilac Lime’ and botanically known as Citrus aurantiifolia. This new lime cultivar was invented in Albany, California and originated from an invention idea Jun. 25, 2012, with bioinformatics research and transgenic promoter testing completed Jun. 2, 2014, with production of the transgenic trees Mar. 25, 2015 and finally confirmation of Mexican lime fruit with anthocyanin expression in the juice sac Jun. 20, 2015. This cultivar is a transgenic tree containing a Citrus tissue-specific promoter (CitWAXp) controlling expression of the blood orange (Moro) CsMybA gene. The present invention has been found to be stable and reproduce true to type through successive asexual propagations.

The present invention relates to a new and distinct cultivar of Mexican lime tree, botanically known as Citrus aurantiifolia Lilac Lime line 9-10, referred to hereafter by its cultivar name, ‘Lilac Lime’.

The new cultivar was derived from a Citrus transformation program in Albany, California, U.S.A. The overall purpose of the transformation program is the creation of transgenic Citrus that exhibit the improved disease resistance, to abiotic and biotic stresses, and fruit quality of various Citrus varieties.

‘Lilac Lime’ was selected as a single unique tree (line 9-10) from greater than 200 that resulted from transformation program made in 2012 with seed derived from Citrus aurantiifolia.

Asexual reproduction of the new cultivar was first accomplished by stem cuttings in Albany, California in 2017. It has been determined that the characteristics of this cultivar are stable and are reproduced true to type in successive generations.

The ‘Lilac Lime’ was developed by transferring the pigment (MybA) gene from the edible Citrus variety, blood orange (Moro) into the Mexican lime genome. The anthocyanin pigment produced by the CsMybA gene in ‘Lilac Lime’ is expressed in the fruit/flower through the use of a tissue-specific promoter (CitWAXp; GenBank MK012380; U.S. Pat. No. 11,739,341). This limits the anthocyanin pigment to the fruit interior and provides an eye-pleasing color purple (juice sacs)/green (peel) combination. Use of the promoter also removes the need for specific climatic growth conditions to produce the coloration. Further, the anthocyanins produced have been linked to the prevention of a number of human health issues. The original Mexican lime seed used for production was obtained from the certified seed source Lyn Citrus Seed, Inc., Arvin, California.

The tissue-specific promoter CitWAXp used in ‘Lilac Lime’ is disclosed in U.S. Pat. No. 11,739,341, which is incorporated herein by reference. U.S. Pat. No. 11,739,341 provides genetic constructs each containing a promotor, including CitWAXp, that is useful in driving fruit-specific expression in plants. Further provided in U.S. Pat. No. 11,739,341 are expression vectors, transgenic plants (including transgenic Mexican lime plants), and plant parts containing such genetic constructs, as well as uses thereof. The transgenic Mexican lime Citrus lines were generated using epicotyl explants protocol as previously described by de Oliveira et al., Plant Cell Rep (2009) 28:387-395 (doi: 10.1007/s00299-008-0646-2), and de Oliveira et al., HortTechnology (2016) 26(3), 278-286 (doi: 10.21273/HORTTECH.26.3.278), each of which is incorporated herein by reference.

The production and evaluation of transgenic Citrus ‘Lilac Lime’, as well as the genetic modification approach used to develop ‘Lilac Lime’, are described in Thomson et al., Citrograph (2022) 13(3): 48-51, which is incorporated herein by reference.

The CitWAXp promoter was fused to the Citrus MybA (CsMybA) transcription factor from the Moro blood orange (i.e., the Ruby gene) in a pCTAGII binary vector T-DNA and Agrobacterium-mediated transformation was used to generate transgenic Mexican lime trees as described in Thilmony et al., Front. Plant Sci. (2022) 13:945738 (doi: 10.3389/fpls. 2022.945738), which is incorporated herein by reference. FIG. 6 is a diagrammatic representation of the binary plasmid pCTAGII-(CitWAX-Ruby).

In one aspect of the disclosure in U.S. Pat. No. 11,739,341, an expression vector containing a genetic construct is provided. As used herein, the term “vector” refers to a nucleic acid molecule capable of transporting another nucleic acid to which it has been linked. Examples of vector include, but are not limited to, plasmid, cosmid, bacteriophage, bacterial artificial chromosome (BAC), yeast artificial chromosome (YAC), or virus that carries exogenous DNA into a cell. One type of vector is a “plasmid”, which refers to a circular double stranded DNA loop into which additional DNA segments can be ligated. As used herein, “plasmid” and “vector” may be used interchangeably as the plasmid is the most commonly used form of vector. The GUS gene (β-glucuronidase gene) was replaced by the Ruby (CsMybAl) coding sequence. A vector may be a binary vector or a T-DNA that comprises the left border and the right border and may include a gene of interest in between. The term “expression vector” as used herein means a vector capable of directing expression of a particular nucleotide sequence in an appropriate host cell. Thus, a vector containing an expression construct is considered an expression vector.

Binary plasmid construction was as previously described in Dasgupta et al., BMC Biotechnol. (2020) 20:43 (doi: 10.1186/s12896-020-00635-w), which is incorporated herein by reference. The Ruby (CsMybAlMoroMybA) genomic sequence including native introns was synthesized (GenScript USA Inc., Piscataway, New Jersey) to remove native restriction sites and to aid in cloning as described in Dasgupta et al., GM Crops Food (2017) 8:85-105 (doi: 10.1080/21645698.2016.1267897), which is incorporated herein by reference. The complete Ruby sequence is available in Supplementary Text S1 of Dasgupta et al., 2017. The nucleotide sequence for the CitWAXp promoter is available from the GenBank under the following accession number MK012380. Each construct is derived from the pCTAGII-GUSPlus binary vector (GenBank MG818373) and contains the CitWAXp promoter used to express Ruby, along with the codA-nptII selection marker for transgenic tissue selection.

Mexican lime (C. aurantifolia) juvenile tissue was used for hypocotyl transformation as previously described in De Oliveira et al., 2009. Certified Mexican lime seed was obtained from Lyn Citrus Seed, Inc., Arvin, California. The seed was removed of its outer seed coat and surface sterilize the by soaking in 20% bleach for 20 min. Seeds were then rinsed three times in sterile water and 3-5 seeds were placed on Citrus seed germination medium (4.4 g/L MS salts with vitamins, 25 g/L sucrose, adjusted to a pH of 5.8 with 1 N NaOH, 8.0 g/L Bacto Agar) and incubated in the dark at 28° C. for 4-5 weeks. Etiolated seedlings that were 12-15 cm long were used for transformation. A 25 mL Agrobacterium culture was prepared in YEP medium with 50 mg/l kanamycin and 200 μM acetosyringone and incubated at 28° C. shaker at 225 rpm overnight. The Agrobacterium culture was then centrifuged at 3,600×g for 10 min at 22° C., the supernatant poured off and the pellet resuspended in liquid inoculation medium (4.4 g/L MS salts with vitamins, 30 g/L sucrose, adjusted to a pH of 5.8 with 1 N NaOH. After autoclaving add, 200 μM acetosyringone, 400 μl myo-inositol 100 mg/L, 1 mg/ml thiamine-HCL, 1 mg/mL pyridoxine, 1 mg/mL nicotinic acid and 1 mg/mL BAP) and adjusted to an OD₆₀₀ of 0.2-0.4.

Young, healthy stems were cut into pieces approximately 10 mm in length with the ends cut with at least 45° angle so that each angle will be in the opposite direction of the other. The segments were incubated in the Agrobacterium suspension for 10 min and then blotted and allowed to dry on sterile filter paper for 5 min. They were then placed on co-cultivation medium [1.21 g/L Broadleaf Tree Basal Medium (B1396-PhytoTechnology Laboratories, Shawnee, Kansas), 30 g/L sucrose, adjusted the pH 5.8 with 1 N NaOH, and then 8.0 g/L Bacto Agar. After autoclaving, 200 μM acetosyringone, 400 μl/L, myo-inositol 100 mg/L thiamine HCL, 1 mg/mL pyridoxine, 1 mg/ml, nicotinic acid, 1 mg/mL glycine 1 mg/L BAP, 1 mg/ml 2.4-D, and 1 mg/mL, NAA-Sigma-Aldrich, St. Louis, Missouri] in sterile petri dishes and kept in the growth chamber at 28° C. for 2 days in the dark. The infected explants were then transferred to selection regeneration medium (SRM; same as the co-cultivation medium with 70 mg/L of kanamycin added). They were incubated for 2 weeks in dark and then transferred to an incubator at 28° C./16 h light and 24° C./8 h dark. The explants were transferred to fresh SRM every 2-3 weeks.

Primary Mexican lime transformants were regenerated on SRM with 50 mg/L kanamycin. Individual green shoots from explants were excised when approximately 10-12 mm in length and grafted onto Mexican lime seeding rootstocks. Once the grafts were established in tissue culture, the plantlets were transferred to soil in a growth chamber at 16/8 light dark cycle and 28° C. under a humidity dome and allowed to grow for another 4 weeks before being transferred to the greenhouse where they were maintained with a mist spray for 2 weeks and then allowed to harden to greenhouse conditions. Greenhouse conditions were 16/8 light dark cycle and 26° C.

The gene expression control element (promoter) CitWAXp was used to keep the pigment production confined to the fruit tissues. The ‘Lilac Lime’ still produces green peel and mature green leaves, and reaches its fruiting stage in a time-frame similar to traditional Mexican lime varieties. Confining pigment production to the inner fruit prevents the plants from wasting their finite energy resources on producing pigment in all their tissues. Transgenic lineages that used a constitutive promoter to produce these pigments throughout all plant tissues grew smaller and produced fewer fruits, thus excluding them from commercial viability. Use of the tissue-specific promoter CitWAXp provides healthier plants that are not “overtaxed” by producing purple pigmentation throughout the plant without benefit, and possibly detriment, to growth.

Phenotypic characterization indicated that the engineered Citrus trees were robust and produced colored fruit without compromising plant health. Anthocyanin production values within the fruit were estimated to be approximately 35% of a typical blood orange (Moro variety). Field testing was used to select lines that were robust for biotic and abiotic stress, maintained the colored fruit and fruit production was similar to Mexican lime control lines. Seven lines were consistent with growth, fruit production, and fruit color. ‘Lilac Lime’ was chosen both for its deep purple fruit color and because it contained four complete transgene copies within a single locus of genome, simplifying the deregulation process. In short, the ‘Lilac Lime’ was selected based on the size/quantity of fruit production, consistent fruit color and because it did not differ significantly in other categories from the wild-type Mexican lime.

Mention of trade names or commercial products is solely for the purpose of providing specific information and does not imply recommendation or endorsement by the United States Department of Agriculture.

### SUMMARY OF THE NEW PLANT

The following are the most outstanding and distinguishing characteristics of ‘Lilac Lime’ when grown under normal horticultural practices in California. First, anthocyanin is expressed in the juice sac of the ‘Lilac Lime’ fruit as compared to Citrus aurantiifolia from which it was derived providing a deep purple color. Second, ‘Lilac Lime’ has a consistent and homogenous anthocyanin expression pattern across the entire fruit as compared to the blood orange (C. sinensis; Moro) from which the CsMybA gene for anthocyanin expression was derived. Third, ‘Lilac Lime’ does not require specialized temperature/weather conditions to induce anthocyanin expression within the juice sac of the fruit as compared to blood orange (C. sinensis; Moro) from which it was derived. Fourth, the ‘Lilac Lime’ is a low seeded variety (i.e., 0-2 seeds per fruit) as compared to Citrus aurantiifolia from which it was derived, which is considered a moderate to highly seeded cultivar (i.e., with up to 15 seeds per fruit, averaging approximately 4-6 seeds per fruit). Low seediness is a prime requirement for most Citrus. As the ‘Lilac Lime’ is a transgenic cultivar it contains kanamycin resistance (nptII), cytosine deaminase (codA) both for tissue selection and the Flowering Time (FT) gene to speed time to maturity/fruit production. ‘Lilac Lime’ has not been observed under all environmental conditions and cultural practices, and some characteristics may differ outside of California or with different management practices.

### BRIEF DESCRIPTION OF THE PHOTOGRAPHS

The accompanying photographs show the flowers, fruit and entire plants; the colors shown are as true as can be reasonably obtained by conventional photographic procedures.

FIG. 1 shows ‘Lilac Lime’ cross section of mature fruit from the field, displaying anthocyanin expression in the juice sac and green peel.

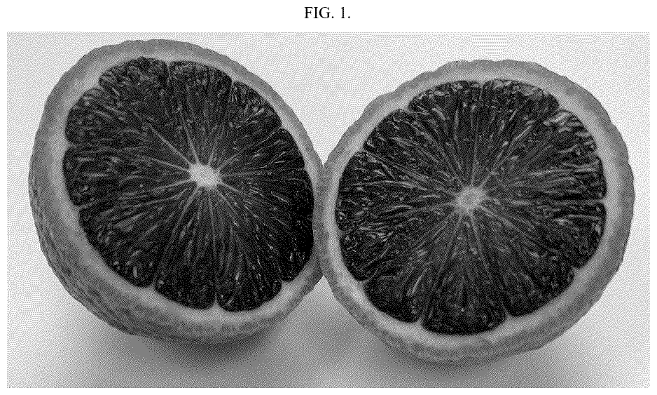

FIG. 2 shows a ‘Lilac Lime’ mature flower from the field, displaying anthocyanin expression in the pistil, stamin, and anther filaments and lighter anthocyanin expression in the petals.

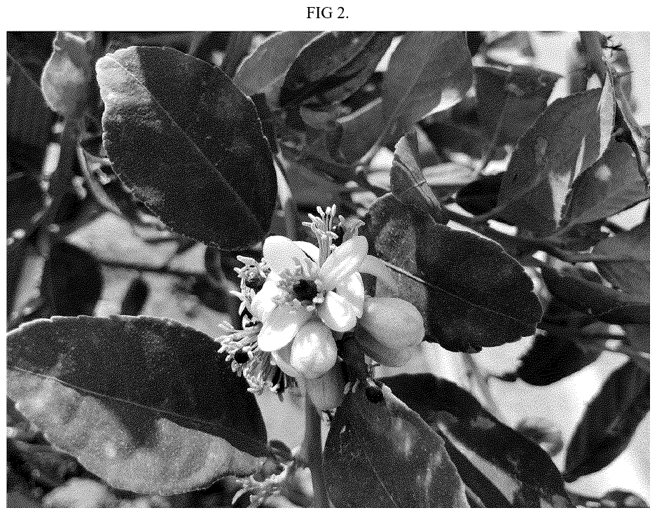

FIG. 3 shows ‘Lilac Lime’ cross section of an immature fruit from the field, displaying anthocyanin expression in the juice sac.

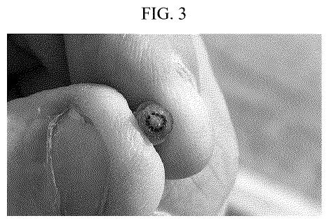

FIG. 4 shows mature and immature fruit on a ‘Lilac Lime’ tree in the greenhouse.

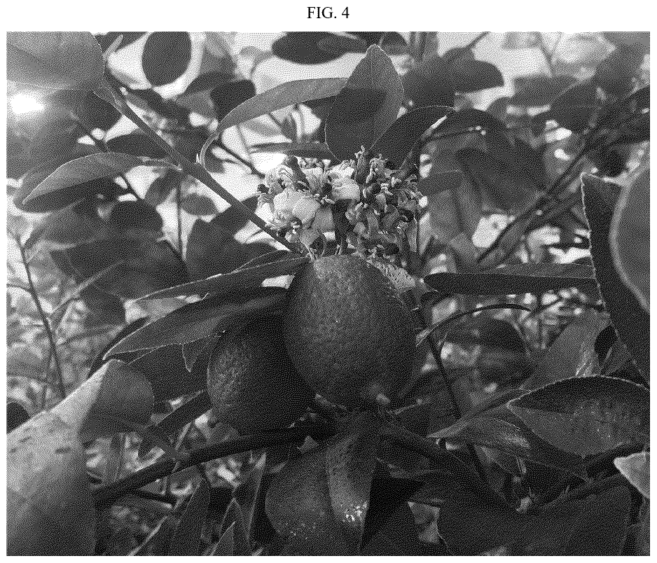

FIG. 5 shows ‘Lilac Lime’ trees in the greenhouse.

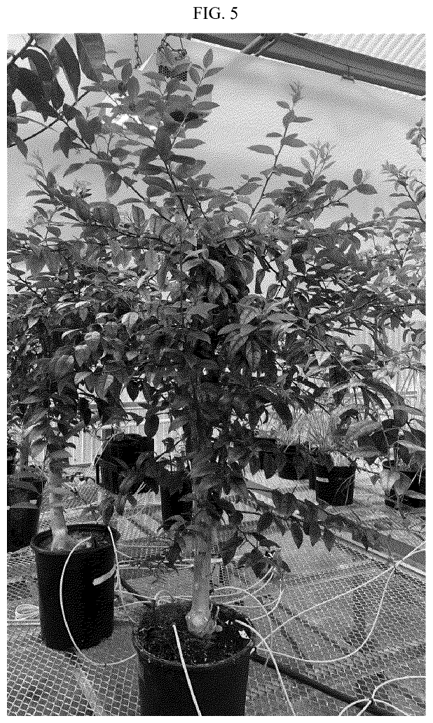

FIG. 6 is a diagrammatic representation of the binary plasmid pCTAGII-(CitWAX-Ruby) used to transform Mexican lime to obtain ‘Lilac Lime’.

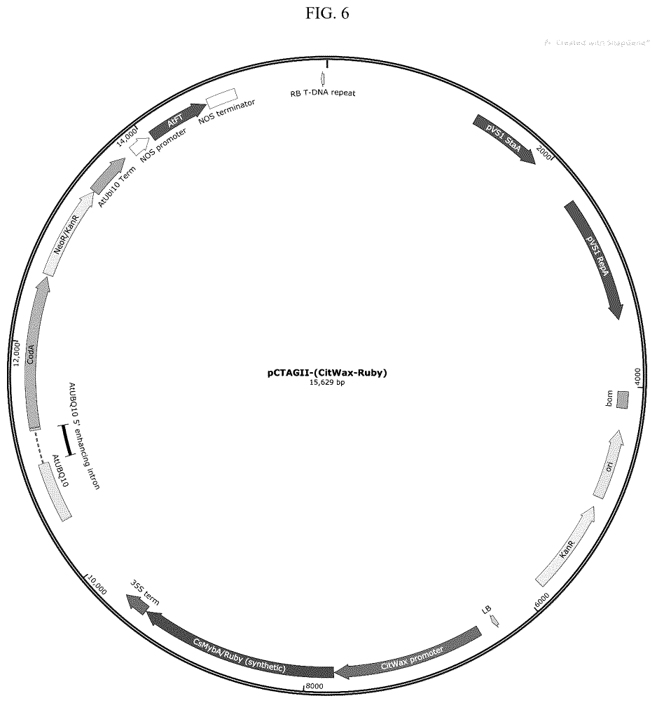

Due to photographical light, chemical development, processing and printing, and copying, the leaves and fruit depicted in these photographs may or may not be accurate when compared to the actual botanical specimen.

### DETAILED DESCRIPTION OF THE NEW CULTIVAR

The following description of ‘Lilac Lime’ is based on observations taken from 2015 to 2024 growing in greenhouse trials in Albany, California and 2018 to 2024 growing field in trials in Exeter, California. This description is in accordance with UPOV terminology. Color designations, color descriptions and other phenotypical descriptions may deviate from the stated values and descriptions depending upon variation in environmental, seasonal, climatic and cultural conditions. ‘Lilac Lime’ has not been observed under all possible environmental conditions. Color terminology follows The Royal Horticultural Society Colour Chart. London (R.H.S.) (5^{th} edition, 2007).

Important plant characteristics of the ‘Lilac Lime’ cultivar are set forth below. Characteristics include physical and chemical composition of the fruit and tree from greenhouse in Albany, California (Lat 37.885535°, Lon −122.303562°) and field studies in Exeter, California (Lat 36.351437°, Lon −119.060418°).

Greenhouse evaluation: A chlorophyll meter (SPAD-502, Spectrum Technologies, Inc. Aurora, Illinois, United States) and leaf porometer (SC-1, Meter Group Inc., Pullman, Washington) were employed to investigate the physiological status of the transgenic trees in direct comparison to wildtype controls. SPAD values are proportional to leaf chlorophyll content as a measure of nitrogen use efficiency. ‘Lilac Lime’ demonstrated a SPAD reading of 40.5 μmol/m² while the Mexican lime control showed a reading of 40.4 μmol/m². The porometer estimates stomatal conductance by measuring water loss from the leaf, which is dependent on the rate of transpiration. The stomatal conductance value for ‘Lilac Lime’ was 220.7 mmol/m²s while the wild type Mexican lime (WT MxLm) control was 223.5 mmol/m²s. Both SPAD and porometer estimates demonstrate the physiological status of the ‘Lilac Lime’ to be similar to the control WT MxLm trees. Another evaluation considered the Lime flavor which is influenced by the balance between sugars and acidity. Therefore, both BRIX and pH were analyzed to determine if the presence of anthocyanin had an effect. ‘Lilac Lime’ showed a BRIX value of 6.94+0.31 while the WT MxLm had a BRIX of 7.31+0.2. Further, ‘Lilac Lime’ had a pH of 2.22+0.06 while the WT MxLm had a pH of 2.05+0.01. These two measurements indicate much more than just flavor, they act as a litmus scale to determine overall fruit quality. Results indicate little to no change in the BRIX and pH values due to the addition of anthocyanin to fruit. Fruit juice compositional analysis was conducted by the USDA-AMS National Science Laboratories from greenhouse grown ‘Lilac Lime’ trees and compared to store bought WT MxLm. Eighteen compounds were analyzed, and results demonstrated that the colored fruit juice had the same nutritional quality as wildtype fruit. Nutritional quality results for the ‘Lilac Lime’ was: Carbohydrate 8.20 g/100 g, Fat 0.90 g/100 g, Moisture 90.4 g/100 g, Protein 0.31 g/100 g, Fructose 0.28 g/100 g, Glucose 0.09 g/100 g, Sucrose 0.01 g/100 g, Vitamin A N.D. mcg/100 g, Vitamin C 19.4 mg/100 g, Calcium 0.29 mg/100 g, Copper 0.01 mg/100 g, Iron 0.07 mg/100 g, Magnesium 5.57 mg/100 g, Phosphorus 2.19 mg/100 g, Potassium 19.3 mg/100 g, Selenium<0.5 mcg/100 g, Sodium 0.36 mg/100 g, Zinc 0.11 mg/100 g. The WT MxLm fruit juice composition was: Carbohydrate 7.80 g/100 g, Fat 0.88 g/100 g, Moisture 90.8 g/100 g, Protein 0.33 g/100 g, Fructose 0.33 g/100 g, Glucose 0.22 g/100 g, Sucrose 0.00 g/100 g, Vitamin A N.D. mcg/100 g, Vitamin C 32.5 mg/100 g, Calcium 0.59 mg/100 g, Copper 0.02 mg/100 g, Iron 0.02 mg/100 g, Magnesium 5.50 mg/100 g, Phosphorus 1.78 mg/100 g, Potassium 16.7 mg/100 g, Selenium<0.5 mcg/100 g, Sodium 0.55 mg/100 g, Zinc 0.10 mg/100 g.

Field evaluation: Whole fruit compositional analysis was again conducted by the USDA-AMS National Science Laboratories from three ‘Lilac Lime’ trees over two consecutive years and compared to a WT MxLm tree grown under identical field conditions. Nineteen compounds were analyzed, and results demonstrated that the colored fruit had the same nutritional quality as wildtype fruit. Results demonstrated that the colored fruit had the same nutritional quality as wildtype fruit. Nutritional quality results for the ‘Lilac Lime’ was: Ash 0.35, Calcium 0.01 mg/100 g, Carbohydrate 12.3 g/100 g, Copper 0.01 mg/100 g, Fat 0.8 g/100 g, Fructose 1.34 g/100 g, Glucose 1.24 g/100 g, Iron 0.12 mg/100 g, Magnesium 1.05 mg/100 g, Moisture 85.7 g/100 g, Phosphorus 2.31 mg/100 g, Potassium 17.79 mg/100 g, Protein 0.9 g/100 g, Selenium N.D. mcg/100 g, Sodium 0.10 mg/100 g, Sucrose 0.50 g/100 g, Fiber 4.46%, Vitamin C 26 mg/100 g, Zinc 26.34 mg/100 g. The WT MxLm whole fruit composition was: Ash 0.41, Calcium 0.01 mg/100 g, Carbohydrate 13.7 g/100 g, Copper 0.02 mg/100 g, Fat 0.9 g/100 g, Fructose 1.16 g/100 g, Glucose 1.12 g/100 g, Iron 0.11 mg/100 g, Magnesium 1.60 mg/100 g, Moisture 84.1 g/100 g, Phosphorus 1.99 mg/100 g, Potassium 19.16 mg/100 g, Protein 0.9 g/100 g, Selenium N.D. mcg/100 g, Sodium 0.14 mg/100 g, Sucrose 0.83 g/100 g, Fiber 5.05%, Vitamin C 20 mg/100 g, Zinc 25.86 mg/100.

Fruit weight, size and shape were also analyzed in 2022 and 2023. In 2022 three out of four trees, for both ‘Lilac Lime’ and the control WT MxLm trees, produced fruit. ‘Lilac Lime’ produced a total lime fruit harvest weight that ranged from 582.4-1273.8 g, individual fruit weight ranged in weight from 34.2-44.5 g, fruit length 39.0-42.5 mm, and fruit width 40.0-41.0 mm. All the ‘Lilac Lime’ fruit had a green semi smooth skin and were round to oval in shape. The control WT MxLm trees produced a total lime fruit harvest weight that ranged from 592.2-1413.8 g, individual fruit weight ranged in weight from 23.8-31.2 g, fruit length 38.9-42.5 mm, and fruit width 34.5-37.8 mm. All the WT MxLm fruit had a green semi smooth skin and were round to oval in appearance.

Four ‘Lilac Lime’ trees were field planted in 2019 and evaluated for size in 2024. All four ‘Lilac Lime’ trees survived the four years. Five WT MxLm control trees were also planted and four trees survived for analysis in 2024. The ‘Lilac Lime’ trees ranged in height from 121.92-137.16 cm, width 162.52-200.66 cm, had a canopy density of 1.76×10⁶-2.47×10⁶ cm³ and a trunk diameter from 23.50-29.21 cm. While the WT MxLm control trees ranged in height from 91.44-137.16 cm, width 124.46-203.20 cm, had a canopy density of 6.58×10⁵-2.15×10⁶ cm³ and a trunk diameter from 23.50-29.21 cm. Results indicate the ‘Lilac Lime’ trees height, width, canopy density and trunk diameter are similar to the WT MxLm control trees.

The following is a detailed description of four year-old plants (from grafted cuttings) of ‘Lilac Lime’ as grown in the field under bee netting in Exeter, California, with measurements of mature trees taken from plants grown in a trial plot for a period of 4 years. The phenotype may vary somewhat with variations in temperature, day length, light intensity, soil types and water and fertility levels, without any variance in the anthocyanin genotype.
Tree description:
   Tree type.—Tropical to Sub-tropical shrubby tree.
   Tree habit.—Broad, upright growth habit.
   Tree size.—Reaches about 10-20 feet in height and 10 feet in width. The four year old ‘Lilac Lime’ were 4 to 4.5 feet in height and 4 feet in width.
   Trunk description.—Multi-stemmed from base, a four year-old plant has an average of 4-6 main stems from base; an average of 25 cm in diameter observed 30 cm above soil level.
   Growth rate.—Moderate.
   Diseases and pests.—No unique susceptibility or resistance has been observed when grown with other Mexican lime, W. navel and tangerines cultivars.
   Hardiness.—At least U.S.D.A. Zone 9-11. The ‘Lilac Lime’ can not tolerate freezing temperatures and starts to incur frost damage at 32 degrees.
   Propagation.—Stem cuttings or budding onto rootstock.
   Branching habit.—Freely branched, branching at approximately 45° from main stems.
   Branch diameter.—Average of 6 cm.
   Branch surface.—Smooth on new growth and becoming sandpaper-like with irregular fine ridges in 3 to 4 years.
   Internode length.—Average of 2.5 cm.
   Branch frequency.—Moderate.
   Branch strength.—Strong.
   Bark color of tree trunk.—Immature green to mature light brown.
Description of mature shoots:
   Stem appearance.—Young stems irregularly angular in shape, filling out to round with age.
   Shoot texture.—Smooth when young and sandpaper-like with irregular fine ridges after 3 years.
   Shoot internode length.—Average of 2.5 cm.
   Stem strength.—Strong and flexible, not brittle.
   Thickness of shoot at center of middle internode.—Variable with age.
   Shine of bark.—Juvenile; smooth, matte finish.
   Stem color.—Green to light brown.
   Shoot angle.—Approximately 45°.
Description of growing shoots:
   Color of shoot.—Deep green.
   Surface.—Smooth.
Leaf description:
   Leaf orientation.—Upward to outward.
   Leaf division.—Simple.
   Leaf shape.—Ovate.
   Leaf blade size.—Average of 9 cm in length and 2.5 cm in width.
   Leaf apex.—Acuminate to cuspidate.
   Leaf base.—Rounded to attenuate.
   Leaf surface.—Slightly glaucous to glossy, speckled with minutely translucent glands.
   Leaf texture.—Thin and leathery.
   Leaf margin.—Smooth, with minute translucent glands.
   Leaf color.—Upper surface emerging leaves light green, mature leaves medium green.
   Leaf venation.—Pinnate, upper surface light green, lower surface. prominently raised main vein and slightly raised lateral veins, similar light green in color.
   Petiole.—Winged.
   Durability of foliage to stress.—Returns from wilt with no damage if watered. Will drop rapidly if desiccated for too long. Resists wind.
   Thorns.—Very thomy, present everywhere, 0.3 to 3 cm in length, 0.5 to 2 mm in diameter.
Flower description:
   Flowering period.—Flowers appear throughout the year, but are most abundant from May to September.
   Number of flowers.—1 to 6 per node.
   Inflorescence type.—Single flowers.
   Flower buds.—Globose in shape, 1 cm in width and 2 cm in width. Light lilac in color.
   Flower size.—2.5 cm in diameter. Light lilac in color.
   Flower fragrance.—Mildly sweet, gardenia-like.
   Flower aspect.—Upright and outward.
   Petals.—5 per flower, un-fused, elliptic in shape, acute apex, truncate base, entire margin, about 2 cm in length and 1 mm in width, color of upper and lower surface light lilac surface is satiny and glabrous, thick substance.
   Sepals.—5, fused with an small acute apex on each, light green in color on upper and lower surface, waxy surface, triangular in shape, entire margin, curled inward around nectary, fused base, calyx is 2.5 mm in diameter, 3.5 mm in length and circular in shape and tapering to a small tube about 3 mm in length and width.
   Pedicel.—Green in color, average of 1.5 mm in length and 1.5 mm in width, waxy surface.
   Pistil.—1.5 mm in length, style is green in color and 4 mm in length, stigma is lilac in color and globose in shape, ovary is globose in shape, inferior, waxy in appearance, nectary is disk-shaped, 3 to 4 mm in diameter, 1 mm in depth, waxy surface.
   Stamens.—16 to 18 per flower, filaments are fused into upright ring, lilac in color, and 8 to 10 mm in length, anther is oblong in shape, yellow color and 1.5 to 2 mm in length, pollen is dry and powdery and cream in color.
Fruit description:
   Fruit harvest.—First picking is generally mid October if temperature have been cool for sufficient sugar development, last picking is generally late November just prior cold temperatures.
   Fruit type.—Hesperidium, average of 11 segments.
   Fruit size.—Average fruit length 38.9-42.5 mm, fruit width 34.5-37.8 mm.
   Fruit weight.—Ranged from 23.8-31.2 g per fruit.
   Fruit aroma.—Skin when peeling; strong limonene oil, flesh; skin green, flesh purple.
   Fruit shape.—Round to Oblate.
   Fruit symmetry.—Nearly symmetrical although it can also be oblong.
   Fruit flavor.—Tart.
   Fruit skin.—Can be peeled, 0.5 cm in thickness, outer surface; rugulose, slightly glossy, deep green. Will ripen to yellow but not desired for marketing.
   Color of outer surface.—Deep green to yellow.
   Color of flesh.—Deep purple when younger with green peel, will lighten to a lilac when fruit matures and peel yellows.
   Color of albedo.—Lilac.
   Navel presence.—None.
   Skin (rind).—Can be peeled, 0.5 cm in thickness, outer surface; rugulose, slightly glossy, deep green to yellow as fruit mature.
   Fruit segments.—Kidney-shaped, an average of 2 cm in length and 1.5 cm in width.
   Fruit set.—Medium, heavily dependent on early spring weather during bloom.
   Seed.—0 to 2 seeds per fruit.
   Texture of flesh.—Juicy, glossy, slight indentations.
   Cropping frequency.—Annually.
   Fruit brix.—6.94+0.31 from refractometer.
   Juice production.—Average of 15-20 ml per fruit.
   Fruit keeping quality.—About 4 weeks at room temperatures before rind begins to mummify and fruit volume begins to shrink from dehydration, 6 to 8 weeks if refrigerated with humidity control.
   Fruit shipping quality.—Thin skinned, but relatively tough; packing materials do not appear necessary.
   Fruit use.—Primarily for fresh consumption, juiced, cooking or decoration.

### COMPARISON WITH PARENTAL AND COMMERCIAL CULTIVARS

‘Lilac Lime’ differs from other commercial Mexican lime plants where the fruit flesh contains anthocyanin and is a deep purple in color while retaining the green peel. Unlike the parent strain the ‘Lilac Lime’ has a low seed count per fruit (i.e., 0-2 seeds per fruit). Mexican lime wild type is very seedy with up to 15 seeds per fruit, averaging approximately 4-6 seeds per fruit. Low seediness is a prime requirement for most Citrus. Unlike the blood orange from which the anthocyanin transcriptional activator (CsMybA) is derived, the ‘Lilac Lime’ does not require a specific set of temperature/weather conditions in order to produce the purple coloration. Further, the unique promoter used to express the CsMybA transcriptional activator provides a consistent homogenous expression pattern of anthocyanin (purple color) within the fruit.

1. A new and distinct variety of lime plant named ‘Lilac Lime’, substantially as illustrated and described herein, characterized by its fruit flesh which contains anthocyanin and is a deep purple color with a green peel.
